(19) United States
(12) Patent Application Publication
(10) Pub. No.: US 2025/0014682 A1
(43) Pub. Date: Jan. 9, 2025

(54) MACHINE LEARNING WITH NEURAL NETWORKS FOR REDUCING BACTERIAL SEQUENCE CONTAMINATION IN PHAGE GENOME CONSTRUCTIONS

(71) Applicant: City University of Hong Kong, Hong Kong (HK)

(72) Inventors: Shuaicheng LI, Hong Kong (HK); Ruohan WANG, Hong Kong (HK); Guangze PAN, Hong Kong (HK)

(21) Appl. No.: 18/762,693

(22) Filed: Jul. 3, 2024

Related U.S. Application Data
(60) Provisional application No. 63/512,273, filed on Jul. 6, 2023.

(51) Int. Cl. G16B 30/00; G16B 40/20; G16B 45/00

(52) U.S. Cl. CPC G16B 30/00; G16B 40/20; G16B 45/00

(57) ABSTRACT

A machine learning (ML) based system with neural networks for reducing bacterial sequence contamination in constructing a phage genome utilizing next-generation sequencing (NGS) data is present. The system includes a phage NGS dataset and includes a NGS reads screener to filter out low-quality reads. An NGS integrator is employed to pre-assemble the filtered reads into contigs. The contigs are then classified using a contig classifier including an autoencoder based on a gapped pattern graph convolutional network (GP-GCN) to identify their origin phage genome so as to minimize a bacterial sequence contamination in the NGS dataset. A graph generator creates a copy-number-aware bipartite conjugate graph from the classified contigs. A phage sequence assembler analyses the graph for assembling the contigs into a potential phage sequence.

### CROSS-REFERENCE TO RELATED APPLICATIONS

The present application claims priority from U.S. provisional patent application Ser. No. 63/512,273 filed Jul. 6, 2023, and the disclosure of which is incorporated herein by reference in its entirety.

### FIELD OF THE INVENTION

The present invention generally relates to the field of machine learning using neural networks. More specifically the present invention relates to reducing bacterial sequence contamination in constructing the genomes of high-quality and confident phages.

### BACKGROUND OF THE INVENTION

Phages, viruses that infect bacteria or archaea, represent the most diverse and abundant biological entities on Earth (Camarillo-Guerrero et al. 2021). They exert a profound influence on microbial communities, orchestrating the balance and dynamics of ecosystems. Phages contribute to maintaining bacterial diversity through bacterial lysis and facilitate horizontal gene transfer between bacteriophages and bacteria, driving their co-evolution. Consequently, phages impact not only individual bacteria but also the broader ecosystem. In addition, phage therapy emerges as a promising alternative to antibiotics in the age of increasing multi-drug resistance. Despite their significance, the understanding of phages remains limited due to difficulties in laboratory cultivation of phages and their hosts.

Recent advancements in next-generation sequencing (NGS) and phage detection algorithms have enabled the computational mining of phage sequences from metagenomic data (Camargo et al. 2023; Akhter, Aziz, and Edwards 2012; Ren et al. 2017; Song et al. 2019; Kieft, Zhou, and Anantharaman 2020; Guo et al. 2021), significantly expanding our knowledge of phage diversity (Camarillo-Guerrero et al. 2021; Nayfach, Páez-Espino, et al. 2021). Conventionally, NGS reads are assembled into contigs, and computational methods identify phage-derived contigs, i.e., genome fragments, by specific features or signals. However, detecting bacteriophage sequences from metagenomic sequencing data is extremely challenging due to the high levels of noise and the complex nature of the data, which contains multiple species.

The metagenomic assembly process, typically resulting in contigs with an N50 length of around 1 kb, often fails to capture the complete genomes of phages, which commonly range from 10-100 kb in length and can exceed 200 kb in certain cases (Al-Shayeb et al. 2020). This substantial fragmentation hampers subsequent analyses and results in phage detection methods yielding phages with various levels of completeness (Nayfach, Camargo, et al. 2021). The frequent sequence ambiguities on phage genomes disrupt assembly contiguity, leading to further fragmentation (Klumpp, Fouts, and Sozhamannan 2012). Consequently, existing phage databases mined from metagenomic data primarily consist of low-quality phage fragments, which pose challenges in understanding their roles in microbial communities (Shkoporov and Hill 2019).

Assembling phage genomes from complex metagenomic data presents inherent challenges. First, the high level of sequence diversity and genetic mosaicism of phage genomes hinder ab initio assembly since novel sequences may evade detection in reference-based strategies (Sutton et al. 2019). Second, temperate phages integrate into bacterial genomes as prophages and replicate with bacteria before entering lytic cycles (Zhang et al. 2022). Consequently, assembling prophages is susceptible to bacterial contamination, and algorithms based on coverage variations between phages and bacteria are ineffective in detecting prophages (Antipov et al. 2020). Third, phage genomes harbor a high frequency of repeated sequences, which typically cause assembly algorithms to break contigs at the boundaries of these repeats (Shkoporov and Hill 2019). Fourth, phages exhibit varied coverage depths in metagenomic data, making it challenging for algorithms to differentiate between uneven coverage and copy numbers (Klumpp, Fouts, and Sozhamannan 2012).

While the advances in sequencing technology and computational methods have significantly expanded the understanding of phage diversity, assembling high-quality phage genomes from metagenomic data remains a complex and challenging task. Therefore, the present invention aims to provide reliable phage assembly tools that can overcome the inherent difficulties presented by metagenomic data.

### SUMMARY OF THE INVENTION

It is an objective of the present invention to provide a system or method to solve the aforementioned technical problems.

In accordance with a first aspect of the present invention, a machine learning (ML) based system with neural networks for reducing bacterial sequence contamination in constructing a phage genome by next-generation sequencing (NGS) dataset is introduced. Particularly the system includes the following components:
a phage NGS dataset;
a NGS reads screener configured to screen the phage NGS dataset and filter out low-quality NGS reads from the NGS dataset;
a NGS integrator configured to pre-assembles the filtered reads into contigs;
a contig classifier comprising an autoencoder based on a gapped pattern graph convolutional network (GP-GCN) for identifying the origin phage genome of the contigs so as to minimize a bacterial sequence contamination in the NGS dataset;
a graph generator configured to generate a copy-number-aware bipartite conjugate graph from the contigs; and
a phage sequence assembler configured to analyze the copy-number-aware bipartite conjugate graph and assemble the contigs into a potential phage sequence.

In accordance with one embodiment of the present invention, the autoencoder is trained with a phage genome dataset, so that the autoencoder is capable of matching the contigs to its origin phage genome.

In accordance with one embodiment of the present invention, the potential phage sequence is a linear or circular genome.

In accordance with one embodiment of the present invention, the copy-number-aware bipartite conjugate graph includes vertices and edges, particularly, the vertices are endpoints of each of the contigs and the edges are overlapping reads between each of the contigs.

In accordance with one embodiment of the present invention, the contig classifier further utilizes sequence homology and motif analysis to enhance the accuracy of identifying the origin phage genome of the contigs.

In accordance with one embodiment of the present invention, the system further includes a database management system configured to store and retrieve the phage NGS dataset, the filtered reads, the contigs, and the assembled phage sequences for future analysis and comparison.

In accordance with one embodiment of the present invention, the NGS reads screener, the NGS integrator, the contig classifier, the graph generator, and the phage sequence assembler are integrated into a unified software platform with a user-friendly graphical interface.

In accordance with one embodiment of the present invention, the system further includes a visualization module that provides graphical representations of the copy-number-aware bipartite conjugate graph and the assembled phage sequences.

In accordance with one embodiment of the present invention, the low-quality NGS reads are characterized by having an average quality score lower than 20, with more than 40% of the bases have a quality score less than 15, with more than 5′N′ bases, or being shorter than 15 bases.

In accordance with a second aspect of the present invention, a method of reducing a bacterial sequence contamination in phage genome constructions using NGS data analyzed by a machine learning system is provided. The method includes the following steps:
inputting a phage NGS dataset and filtering out low-quality NGS reads from the phage NGS dataset;
assembling the filtered reads into contigs;
training a ML model with a phage genome dataset and a bacteria genome dataset such that the trained ML model is able to identify and classify an origin phage genome of the contigs and reducing a bacterial sequence contamination in the phage NGS dataset;
generating a copy-number-aware bipartite conjugate graph based on the classified contigs; and
analyzing the copy-number-aware bipartite conjugate graph so as to assemble the contigs into potential phage sequences.

In accordance with one embodiment of the present invention, the ML model comprises an autoencoder based on a GP-GCN.

In accordance with one embodiment of the present invention, the ML model is trained with sequence homology and motif analysis features to enhance the accuracy of identifying and classifying the origin phage genome of the contigs.

In accordance with one embodiment of the present invention, the potential phage sequence is a linear or circular genome.

In accordance with one embodiment of the present invention, the copy-number-aware bipartite conjugate graph includes vertices and edges, wherein the vertices are endpoints of each of the contigs and the edges are overlapping reads between each of the contigs.

In accordance with one embodiment of the present invention, the method further includes storing and retrieving the phage NGS dataset, the filtered reads, the contigs, and the assembled phage sequences in a database for future analysis and comparison.

In accordance with one embodiment of the present invention, the method further includes visualizing the copy-number-aware bipartite conjugate graph, and the assembled phage sequences through a graphical interface.

In accordance with one embodiment of the present invention, the method further includes a step of fine-tuning the assembled phage sequences by comparing them against known phage databases and making adjustments to improve sequence accuracy.

In accordance with one embodiment of the present invention, the low-quality NGS reads are characterized by having an average quality score lower than 20, with more than 40% of the bases have a quality score less than 15, with more than 5′N′ bases, or being shorter than 15 bases.

### BRIEF DESCRIPTION OF THE DRAWINGS

Embodiments of the invention are described in more details hereinafter with reference to the drawings, in which:

FIG. 1 depicts a block diagram of a system according to one embodiment of the present invention;

FIGS. 2A-2D depict a workflow of a method according to one embodiment of the present invention, in which FIG. 2A shows the step of receiving the input of NGS data and performing quality control and contig pre-assembly, FIG. 2B depicts the contig annotation and filtering, FIG. 2C depicts applying an iterative maximum matching algorithm for assembling a phage-like contigs to the potential phage sequences, and FIG. 2D depicts a filtering criterion to the obtained sequences;

FIGS. 3A-3F depict a phage assembly performance comparison among the present invention and 7 different benchmark methods, in which FIG. 3A presents the number of phage assemblies aligned to each reference, FIG. 3B shows the F1 score, FIG. 3C shows the alignment recall of phage assemblies, FIG. 3D depicts the alignment precision, FIG. 3E displays the reference coverage and FIG. 3F shows the phage assembly coverage;

FIGS. 4A-4F depict the comparison of phages assembled from ERP000108 among the present invention and 7 different benchmark methods, in which FIG. 4A displays the distributions of length, FIG. 4B presents the percentages of prophages, FIG. 4C demonstrates the percentages of viral genes, FIG. 4D depicts the completeness, FIG. 4E shows the percentages of complete and high-quality phages of the detected phages for all the methods, and FIG. 4F shows the cumulative bar plot displaying the quality reports of the detected phages for all the methods;

FIGS. 5A-5H show the evaluations and analyses of phages assembled from metagenomes, in which FIG. 5A depicts the percentage of prophages, the percentage of viral genes, the completeness, the percentage of complete and high-quality phages, and the quality distributions of the phages, FIG. 5B displays the genome size distributions of the phages from the three metagenome studies, FIG. 5C demonstrates the taxonomy classification of phages with the left nodes representing the phages assembled from the three metagenome studies, the right nodes representing the taxonomy classifications, and the edge widths indicating the number of phages assigned to the taxonomies, FIG. 5D present the GO enrichment analysis for phage genes from ERP000108, FIG. 5E present the GO enrichment analysis for phage genes from ERP002061, FIG. 5F present the GO enrichment analysis for phage genes from ERP003612, FIG. 5G depicts the relationship graphs of gene functional classifications, and FIG. 5H the distributions of the number of tRNAs in each phage genome from the three metagenome studies;

FIGS. 6A-6C depict the largest phage genome assembled from human metagenomes, in which FIG. 6A depicts the largest phage genome assembled from ERP000108 study, FIG. 6B shows the largest phage genome assembled from ERP002061 study, and FIG. 6C displays the largest phage genome assembled from ERP003612 study;

FIGS. 7A-7E depict the phage-host interaction investigation of the phages assembled from ERP000108, ERP002061, and ERP00361, in which FIG. 7A is the host phylogenetic tree of phage assembled from the three metagenome studies, FIG. 7B presents the number of CRISPR arrays detected on phage genomes assembled from the three metagenome studies, FIG. 7C displays the length distribution of CRISPRs detected from the phages, with the inset providing a zoomed-in profile, FIG. 7D shows the repeat length distribution of CRISPRs detected from phages, with the inset providing a zoomed-in profile, and FIG. 7E is the heatmap of anti-CRISPR genes detected from the phages;

FIGS. 8A-8E depict the analyses of phages assembled from metagenomes of healthy controls and CRC patients, in which FIG. 8A displays the percentage of prophages, the percentage of viral genes, the completeness, the percentage of complete and high-quality phages, and the quality distributions of the phages, FIG. 8B shows the host phylogenetic tree of phages assembled from the two metagenome studies, FIG. 8C is the heatmap of virulent factors detected from the phages, FIG. 8D shows the KEGG pathway enrichment analysis for phage genes from CRC patients, with phage genes from healthy controls and adenoma patients as background, and FIG. 8E depicts the GO enrichment analysis for phage genes from FengQ_2015 and YuJ_2015;

FIGS. 9A-9E depict the genome size distributions of the assembled phages from different groups and the number of contigs assembled in each large phage genome, in which FIG. 9A is from the healthy controls and adenoma patients in FengQ_2015, FIG. 9B is from the CRC patients in FengQ_2015, FIG. 9C is from the healthy controls in YuJ_2015, and FIG. 9D is from the CRC patients in YuJ_2015, FIG. 9E depicts the number of assembled contigs;

### DETAILED DESCRIPTION

In the following description, systems and/or methods of constructing high-quality phage genome and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “next-generation sequencing” refers to a suite of modern sequencing technologies that have revolutionized genomic research by allowing the rapid sequencing of entire genomes or targeted regions. The principle behind NGS is based on massively parallel sequencing, where millions of small fragments of DNA are sequenced simultaneously, producing vast amounts of data in a relatively short time.

As used herein, the term “bacterial contamination” in the context of phage genome assembly from metagenomic data refers to the presence of bacterial genetic material that interferes with the accurate assembly and analysis of phage genomes. This contamination arises because temperate phages can integrate into bacterial genomes as prophages and replicate along with bacterial DNA. When attempting to assemble phage genomes, the algorithms can mistakenly incorporate bacterial sequences, leading to errors and inaccuracies. This issue is compounded by the inherent challenges of high sequence diversity, genetic mosaicism of phage genomes, frequent repeated sequences, and varying coverage depths in metagenomic samples.

In accordance with a first aspect of the present invention, a machine learning (ML) based system with neural networks for reducing bacterial sequence contamination in constructing a phage genome utilizing a next-generation sequencing (NGS) dataset is provided. This advanced system is designed to streamline the process of genome assembly from phage NGS data, enhancing accuracy and efficiency through the integration of multiple specialized components.

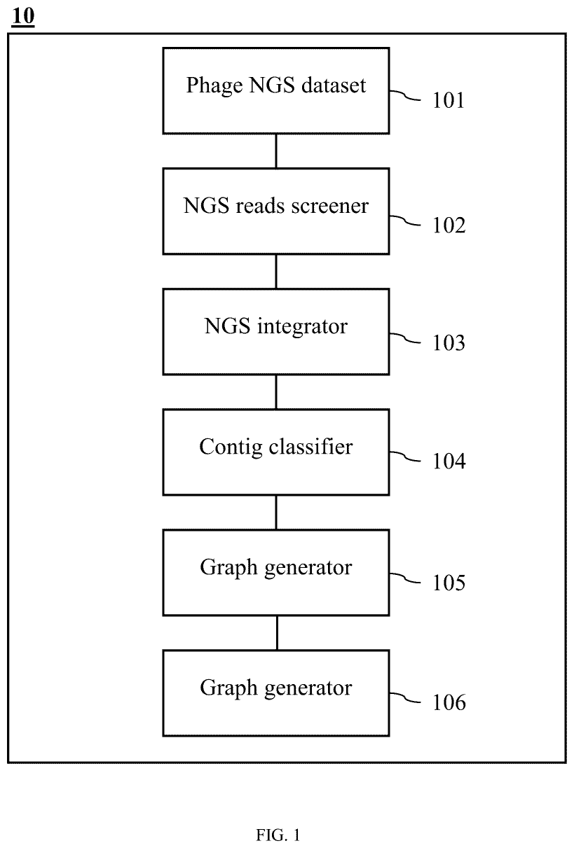

Referring to FIG. 1, the machine learning based system 10 begins with a phage NGS dataset 101, which serves as the foundational input. To ensure data quality, a NGS reads screener 102 is employed to meticulously screen the dataset, filtering out low-quality NGS reads that may compromise the integrity of the subsequent assembly process. This screening is crucial for maintaining high accuracy in the data used for genome construction.

Once the high-quality reads are isolated, the NGS integrator 103 pre-assembles these filtered reads into contigs. These contigs represent contiguous sequences of DNA that are pieced together from overlapping NGS reads, forming the building blocks of the genome assembly.

A pivotal component of the system is the contig classifier 104, which has an autoencoder (not shown) based on a gapped pattern graph convolutional network (GP-GCN). This classifier is specifically trained to identify the origin phage genome of the contigs for minimizing a bacterial sequence contamination in the NGS dataset. The autoencoder's training involves a phage genome dataset, enabling it to match the contigs to their respective origin phage genomes accurately. This classification process is further refined by utilizing sequence homology and motif analysis, enhancing the accuracy of identifying the origin phage genome of the contigs.

Following classification, a graph generator 105 takes over, generating a copy-number-aware bipartite conjugate graph from the contigs. This graph includes vertices and edges, where the vertices represent the endpoints of each contig and the edges denote overlapping reads between the contigs. The graph accurately reflects variations in copy number, which is essential for the correct assembly of repetitive or duplicated regions within the genome.

The final assembly step is managed by a phage sequence assembler 106. This assembler 106 analyzes the copy-number-aware bipartite conjugate graph and systematically combine the contigs into a potential phage sequence, ensuring accurate reconstruction of the phage genome by considering copy number variations and overlaps. The resulting genome sequence can be either linear or circular, depending on the nature of the phage genome being constructed.

In some embodiments, the phage sequence assembler 106 applies an iterative maximum matching algorithm to the copy-number-aware bipartite conjugate graph for a better analyzation. This algorithm is derived from the Hungarian algorithm and has been improved into an iterative process and adapted to fit the conjugate graph data structure.

To support the entire process, the machine learning based system 10 may further includes a database management system. This system is configured to store and retrieve the phage NGS dataset, filtered reads, contigs, and the assembled phage sequences, facilitating future analysis and comparison. This ensures that all data is organized and accessible for subsequent research or verification purposes.

The system machine learning based 10 is designed for user convenience, integrating the NGS reads screener, NGS integrator, contig classifier, graph generator, and phage sequence assembler into a unified software platform. This platform features a user-friendly graphical interface, making it accessible for users with varying levels of technical expertise.

Moreover, a visualization module is incorporated into the machine learning based system 10. This module provides graphical representations of the copy-number-aware bipartite conjugate graph and the assembled phage sequences. These visualizations aid researchers in understanding the assembly process and verifying the accuracy of the constructed genome.

In some embodiments, the low-quality NGS reads are characterized by having an average quality score lower than 20, with more than 40% of the bases have a quality score less than 15, with more than 5′N′ bases, or being shorter than 15 bases.

In one embodiment, to build a comprehensive training dataset, 116,503 high-quality phage sequences are collected from NCBI RefSeq, GPD, MGV, and TemPhD datasets; references of 15,549 bacterial species are downloaded from NCBI RefSeq database. Then, sequence fragments are randomly sampled from the phage and bacterial sequences with random lengths between 50 bp and 5.5 kb, which is the general length range of the SPAdes-generated contigs. Totally 116,445 phage sequence fragments and 108,610 bacterial sequence fragments are obtained, 90% of which are used for training and 10% are used for validation.

Next, the GP-GCN framework is applied to classify the sequences. The framework presents each sequence S as a gapped-pattern graph G (S, k, d), with each k-mer as a vertex and the connection with gaps (distance=d) between two k-mers as an edge. For two vertives a, b ∈ G (S, k, d) (i.e. k-mers Sₐ and S_{b}), an edge from a to b is established if and only if S_{b} appears within distance d following Sₐ. Then, the framework employs l graph convolution layers to learn the interactions between the k-mers and transfer each graph into a latent vector, which is subsequently fed into a neural network for the final prediction. Cross-entropy loss function and Adam gradient descent algorithm are applied for training.

The bacterial lengths are usually hundreds of times longer than phage lengths. Sampling a few (˜7) sequence fragments from each bacterial genome is insufficient to capture the intricate genomic contents of bacteria, which leads to a bias in model training. Thus, a data reconstruction step is adopted to retrain the model. Specifically, 100 fragments are randomly sampled from each bacterial reference and input them into the initial model. If the false-positive rate exceeds 15%, the misclassified samples are included in the negative set. With the reconstructed training set, the model is trained as described above. The results present a significant performance improvement of the retrained model. It is also found that our model outperforms conventional machine learning models and is robust to hyperparameter settings. The system annotates a contig as originating from phage if the softmax score ≥0.7.

In summary, this ML-based system for constructing a phage genome from NGS data represents a significant advancement in the field of genomics. By integrating sophisticated machine learning techniques, high-quality data screening, and efficient assembly algorithms, the system provides a robust and user-friendly tool for accurate phage genome construction.

In accordance with a second aspect of the present invention, a method for reducing bacterial sequence contamination in phage genome constructions utilizing NGS data analyzed by a machine learning system is present. The method is designed to ensure high accuracy and efficiency in assembling phage genomes from sequencing data, leveraging advanced ML techniques and data processing algorithms.

The method begins with inputting a phage NGS dataset. This dataset serves as the initial raw data for the genome construction process. To maintain data integrity and accuracy, the method includes a crucial step of filtering out low-quality NGS reads from the phage NGS dataset. This filtering process ensures that only high-quality reads are used in subsequent steps, thereby enhancing the reliability of the final assembled genome.

Once the high-quality reads are isolated, the next step involves assembling these filtered reads into contigs. Contigs are contiguous sequences of DNA that are formed by piecing together overlapping NGS reads. These contigs serve as the building blocks for the genome assembly process.

Following the assembly of contigs, the method involves training a ML model with a phage genome dataset and a bacteria genome dataset. The trained ML model, which includes an autoencoder based on a GP-GCN, is capable of identifying and classifying the origin phage genome of the contigs and reducing a bacterial sequence contamination in the phage NGS dataset. The training process incorporates sequence homology and motif analysis features to enhance the accuracy of the model, enabling it to accurately match contigs to their respective origin phage genomes.

Subsequent to the classification of contigs, the method involves generating a copy-number-aware bipartite conjugate graph based on the classified contigs. This graph is present with vertices and edges, where the vertices represent the endpoints of each contig and the edges denote overlapping reads between the contigs. The graph accurately reflects variations in copy number, which is essential for correctly assembling repetitive or duplicated regions within the genome.

The copy-number-aware bipartite conjugate graph is further analyzed for assembling the contigs into potential phage sequences. The resulting genome sequences can be either linear or circular, depending on the nature of the phage genome being constructed.

To support the overall process, the method includes storing and retrieving the phage NGS dataset, the filtered reads, the contigs, and the assembled phage sequences in a database. This facilitates future analysis and comparison, ensuring that all data is organized and accessible for subsequent research or verification purposes.

Moreover, the method includes visualizing the copy-number-aware bipartite conjugate graph and the assembled phage sequences through a graphical interface. This visualization aids researchers in understanding the assembly process and verifying the accuracy of the constructed genome.

Additionally, the method includes a step of fine-tuning the assembled phage sequences by comparing them against known phage databases. This comparison allows for making necessary adjustments to improve sequence accuracy, ensuring the reliability of the assembled genome.

In some embodiments, the low-quality NGS reads are characterized by having an average quality score lower than 20, with more than 40% of the bases have a quality score less than 15, with more than 5′N′ bases, or being shorter than 15 bases.

In summary, the present method for constructing phage genomes from NGS data using a ML model represents a significant advancement in the field of genomics. By integrating sophisticated machine learning techniques, high-quality data screening, and efficient assembly algorithms, the method provides a robust and reliable approach to accurate phage genome construction.

As used herein, the term “unified software platform” refers to an integrated system that combines multiple software tools and functionalities into a single cohesive environment, allowing users to seamlessly perform a variety of tasks without needing to switch between different applications. This platform is designed to streamline workflows, improve efficiency, and enhance user experience by providing a centralized interface and consistent user experience across different tools and modules.

As used herein, the term “visualization module” refers to a component of software or a system designed to create visual representations of data. It converts raw data into graphical formats such as charts, graphs, maps, and other visual aids, making complex information more understandable and accessible. The primary goal of a visualization module is to help users interpret data quickly and effectively, identify patterns, trends, and outliers, and support decision-making processes.

In one embodiment, with the NGS pair-end reads from metagenomic sequencing as input, the method first performs quality control with fastp to obtain high-quality reads. Then, the method assembles the filtered reads into contigs with SPAdes using the meta flag. To identify contig candidates from phages, the method incorporates three methods to discriminate whether a contig originates from a phage; these include comparison of phage reference, annotation of phage-specific genes, and scoring of the phage classification model.

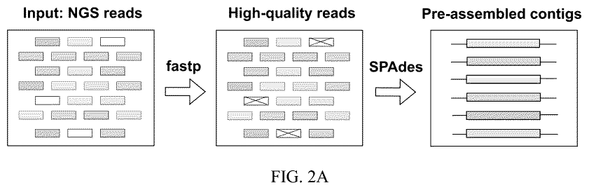

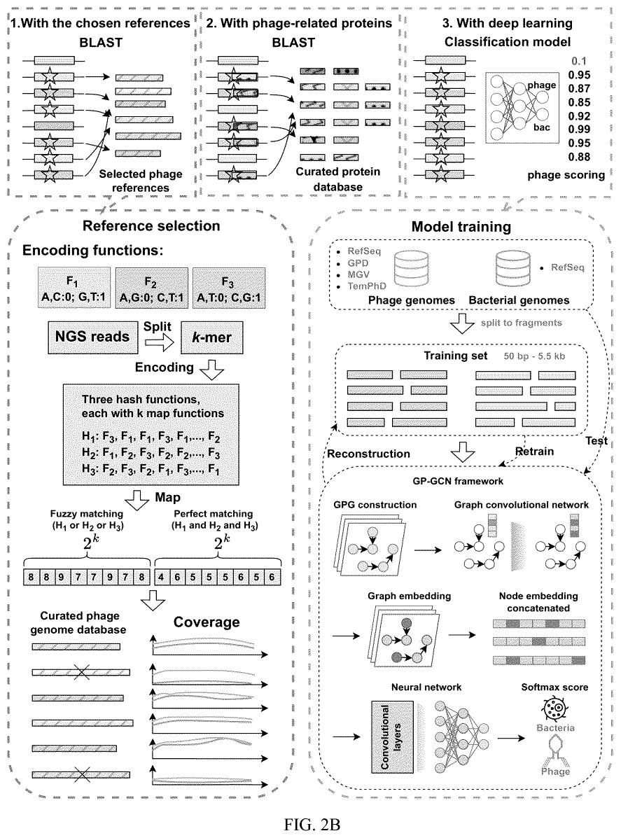

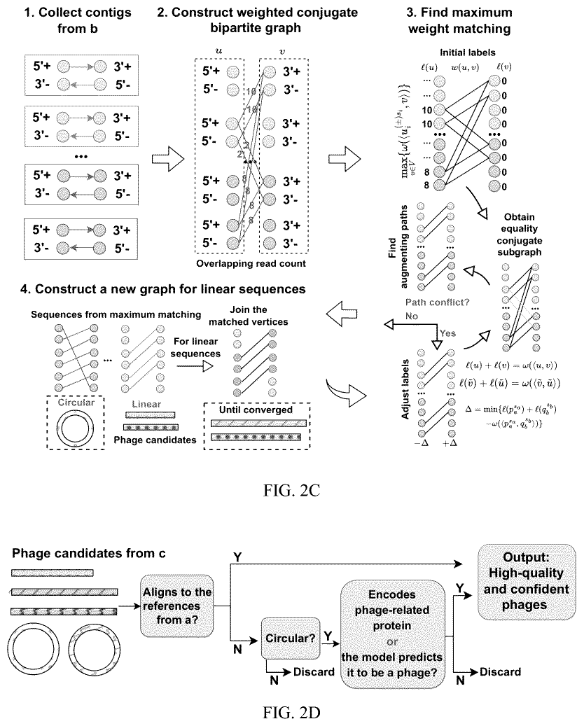

In one embodiment, the method begins with inputting a phage NGS dataset and filtering out low-quality NGS reads from it, followed with pre-assembling the filtered reads into contigs (FIG. 2A). Next, the next step is to integrate homology search and protein annotation. As shown in FIG. 2B, a deep-learning model based on a GP-GCN identifies the origin phage genome of the contigs and piles up the classified contigs for subsequent assembly. Additionally, a copy-number-aware bipartite conjugate graph from the collected contigs is constructed with an iterative maximum matching algorithm for assembling the contigs into potential phage sequences (FIG. 2C). Through this process, the phage fragments are successfully amalgamated, resulting in high-quality phage sequences with linear or circular genomes. For evaluating the quality and the potential of the generated sequences, a meticulous filtering criterion is applied, ensuring the acquisition of reliable and confident phage assemblies (FIG. 2D).

In one embodiment, the method compares the contigs to a curated phage reference database (Nayfach, Camargo, et al. 2021), which includes 58,670 phage genomes, to annotate the contigs derived from phages. To mitigate the computational burden, the method employs a two-step procedure; the first step is to perform a raw but fast match to select candidate genome references that may harbor subsequences similar to the assembled contigs, and the second step is to perform a fine-scale alignment to annotate the contigs.

The method employs a fuzzy k-mer matching algorithm to identify the reference genomes similar to the contigs efficiently. The method first parses the filtered reads into k-mers (k=32). Then, to tolerate mutations and reduce false matches, it encodes each k-mer DNA fragment into three binary strings. The method employs three encoding functions {Fᵢ: {A, C, G, T}→{0,1}|i=1,2,3} to transform a nuclear acid type into a bit. F₁ encodes A and C as 0, G and T as 1; F₂ encodes A and G as 0, C and T as 1; F₃ encodes A and T as 0, C and G as 1. Subsequently, the method constructs three hash functions H₁, H₂, and H₃; each encodes a k-mer m=m₁m₂. . . mₖ, mᵢ ∈ A, C, G, T into a binary string Hᵢ(m)=[Fᵢ_{₁}, (m₁), Fᵢ_{₂}(m₁), . . . , Fᵢ_{ₖ}(m₁)], where Fᵢ_{₁}, Fᵢ_{₂}, . . . , Fᵢ_{ₖ} are randomly chosen from {F₁, F₂, F₃} and the three functions Fᵢ_{ⱼ} of each j are mutually different, 1≤j≤k. A combination of the three non-injective functions H₁: m→v₁, H₂: m→v₂, and H₃: m→v₃, gives an injection H=[H₁, H₂, H₃] to encode a k-mer m into three integers v=[v₁, v₂, v₃], i.e. H: m [symbol] v.

Then, the method uses a sliding window of 500 bp to traverse along the references with a step size of 1 bp. Each k-mer in the sliding window is also mapped to v′=[v′₁, v′₂, v′₃] with function H. A perfect matching between the k-mer from reads and the k-mer from the reference is recorded if v=v′. A fuzzy matching is recorded if there exists i ∈ {1,2,3}, s.t. vᵢ=vᵢ′, which tolerates any single-nucleotide polymorphism (SNP). Denote the number of k-mers from the window as n, the number of k-mers with perfect matching as nₚ, and the number of k-mers with fuzzy matching as n_{f}. If nₚ/n≥θₚ and n_{f}/n≥θ_{f}, the method considers that the reads support the sequence within the window. Herein, θ_{g} and θ_{f} are set as 0.85 and 0.9, respectively. For a reference to be selected, the method requires that the supported length be longer than 70% of the entire reference length.

After selecting the potential references, the method aligns the assembled contigs to the selected references with BLAST. A contig is annotated as aligned to the references if >70% of the contig length is aligned or the aligned sequence is longer than 2 kb.

In addition, the method employs a deep learning model to infer the probability that a contig originates from phage. To resolve the variable lengths and genomic variations of the contigs from metagenomic data, a gapped pattern graph convolutional network (GP-GCN) framework is adopted to train a model to classify the contigs with phage or bacterial origin.

### EXAMPLES

### Example 1. Comparison of Phage Assembly Performance

Simulation experiments are performed to evaluate the method according to one embodiment of the present invention and compared it to seven benchmark methods, including Metaviral SPAdes, geNomad, PhiSpy, VirFinder, Prophage Hunter, VIBRANT, and VirSorter2 (Benchmark methods subsection). To simulate metagenomic data, the phage and bacterial references are sampled from the NCBI RefSeq database and generated NGS reads accordingly with different coverage depths for phages and bacteria. The phage abundance and lifestyles are varied with virulent phages presented as circular genomes and temperate phages randomly integrated into bacterial genomes. Nine simulation scenarios are analyzed, each including five replicates (Table 1). In each replicate, various genomic variations are introduced, including insertions, deletions, duplications, and inversions, into the reference genomes. Subsequently, all the methods are applied to the metagenomic sample from each replicate.

TABLE 1

| | # of | # of | | Phage | Bacteria |
|---|---|---|---|---|---|
| | phages | bacteria | Phage types | depth | depth |
| Scenario 1 | 5 | 100 | Virulent phage | 120 | 60 |
| Scenario 2 | 10 | 100 | Virulent phage | 120 | 60 |
| Scenario 3 | 15 | 100 | Virulent phage | 120 | 60 |
| Scenario 4 | 5 | 100 | Temperate phage | 60 | 60 |
| Scenario 5 | 10 | 100 | Temperate phage | 60 | 60 |
| Scenario 6 | 15 | 100 | Temperate phage | 60 | 60 |
| Scenario 7 | 5 | 100 | Virulent phage | 120 &60 | 60 |
| | | | &Temperate phage | | |
| Scenario 8 | 10 | 100 | Virulent phage | 120 &60 | 60 |
| | | | &Temperate phage | | |
| Scenario 9 | 15 | 100 | Virulent phage | 120 &60 | 60 |
| | | | &Temperate phage | | |

Among these methods, the present method amalgamates the metagenomic contigs into phage assemblies, whereas Metaviral SPAdes directly assembles phage genomes from NGS reads. As for other benchmark methods, they detect phages on single metagenomic contigs, which potentially fragment phage genomes. On average, each phage generated by the present invention is assembled from six to sixteen metagenomic contigs, demonstrating that phages are prone to be assembled into fragments during the initial assembly process and the benchmark methods based on pre-assembled metagenomic contigs fail to generate high-quality phages. QUAST (Gurevich et al. 2013) is further applied to assess the phage assemblies from the present method and benchmark methods.

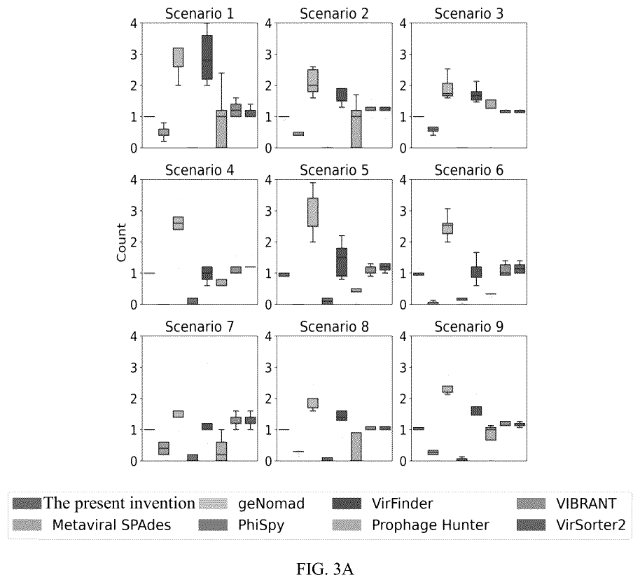

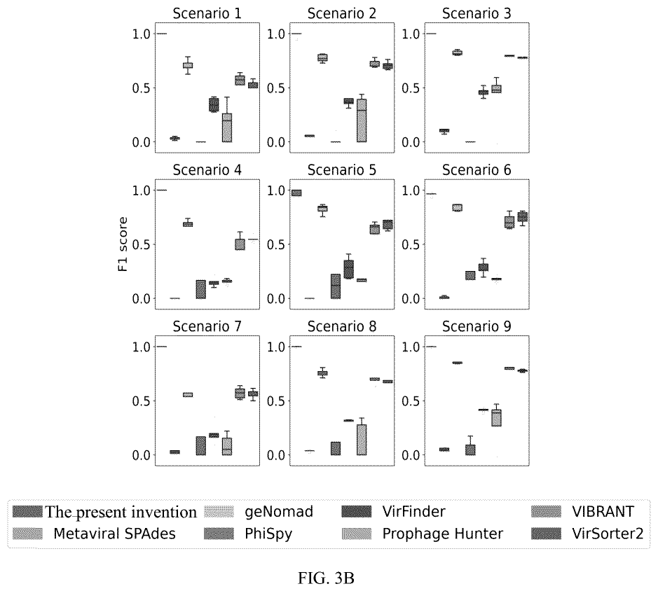

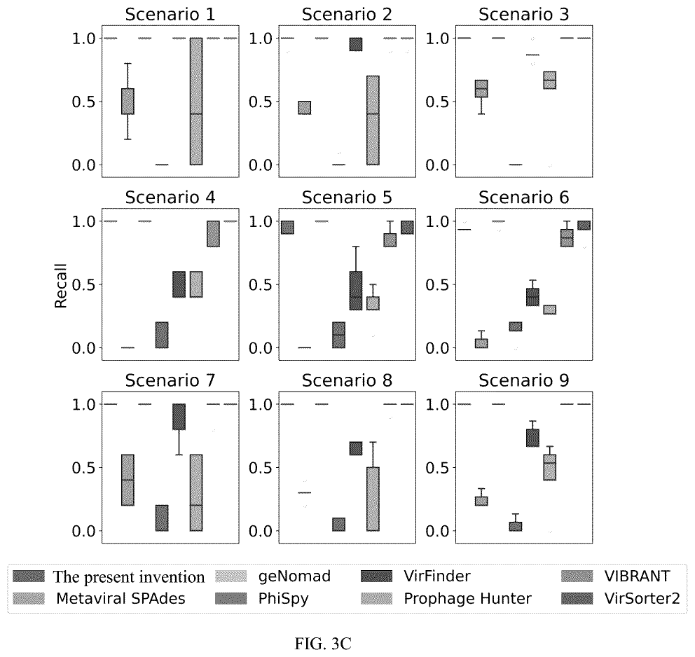

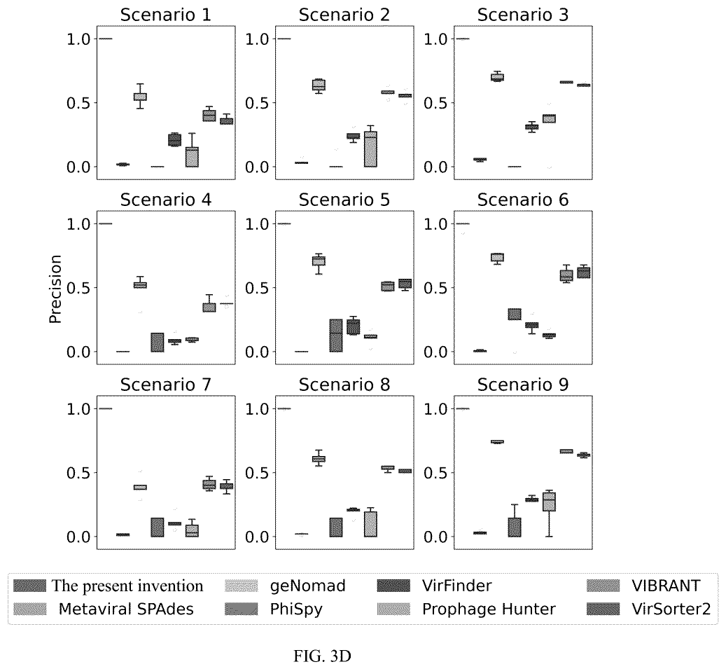

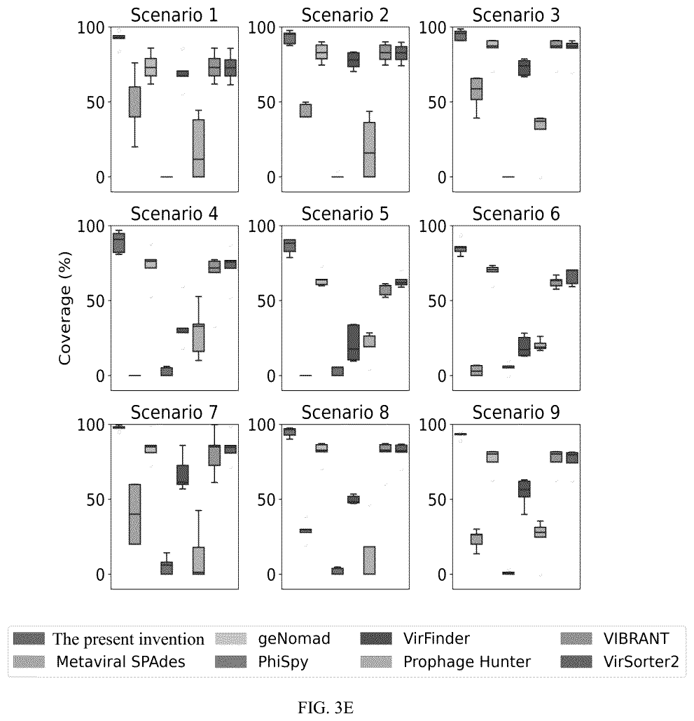

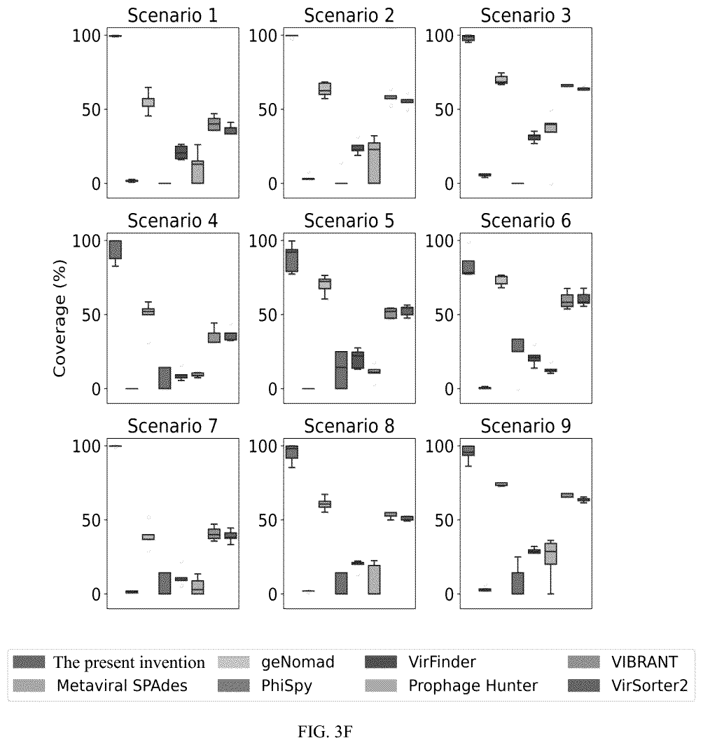

As shown in FIG. 3A, the average number of phage assemblies aligned to each reference among the methods are investigated. A value less than one indicates the failure to capture some phages, while a value larger than one indicates potential fragmentation of the phage genomes. The present invention demonstrates a nearly perfect assembly count, with a median value of one for all nine scenarios, suggesting its precise detection of phage signals and high level of assembly contiguity. VIBRANT and VirSorter2 achieve the second-best performance, with median values of 1.0-1.3 and 1.1-1.4, respectively. Metaviral SPAdes and PhiSpy display median values of 0-0.6 and 0-0.2, respectively, suggesting a greater probability of missing phage signals. geNomad, VirFinder and Prophage Hunter exhibit high variances for the assembly counts, indicating unstable performance.

Referring to FIG. 3B, these methods' F1 scores across the nine scenarios are compared. The present method achieves F1 scores ranging from 0.97 to 1.00,significantly improving the performance of the second-best method by 11.80-85.71%. Moreover, the present method consistently achieves superior recall values of 0.93-1.00 and precision values of 1.00 across the settings (FIG. 3C and FIG. 3D). While geNomad, VIBRANT and VirSorter2 also exhibit high recall values, they experience a noticeable decline in precision, with the median precision values of 0.37-0.76 for geNomad, 0.37-0.68 for VIBRANT and 0.37-0.63 for VirSorter2, suggesting that they misclassify the bacterial sequences as phages.

Referring to FIG. 3E and FIG. 3F, the alignment coverage between phage references and assemblies is examined by quantifying both reference coverage and phage assembly coverage to further evaluate the completeness of phage assemblies. The results indicate that the phage genomes derived by PALACE achieve the highest reference coverage with median coverage values ranging from 85.32% to 97.70%, presenting 8.43-24.44% improvements compared to the second-best method. Specifically, Metaviral SPAdes relies on the coverage depth variations between phages and bacteria (Antipov et al. 2020), which compromises its performance on temperate phages. For these benchmark methods, when all assembled phage fragments are aligned with references, a higher reference coverage is displayed, implying that the phages identified by these methods are predominantly incomplete fragments. Furthermore, the present method obtains the highest phage assembly coverage across the nine scenarios, achieving median coverage values of 78.15-99.82%. In particular, for scenarios 4-6, where only temperate phages are present, distinguishing the phages becomes more challenging since temperate phages are integrated into bacterial genomes. In these cases, the present method still outperforms benchmark methods with a coverage improvement of 2.51-47.68% compared to the second-best method. These results indicate that the present method generates more contiguous and precise phage assemblies.

### Example 2. Evaluation of the Present Method on NGS-Based Metagenomic Data

The present method and the seven benchmark methods (Benchmark methods subsection) are applied to 124 human gut metagenomic samples from the study ERP000108 (Qin et al. 2010) and examined the generated phage genomes. Herein, 156 human gut metagenomes of individuals from Denmark and Spain are utilized.

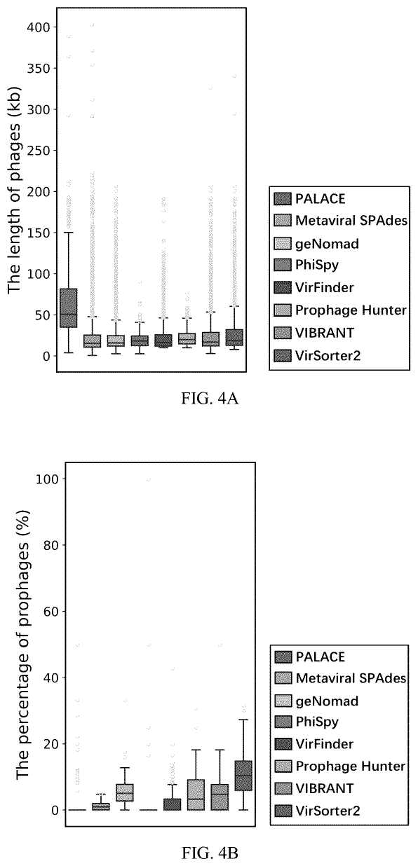

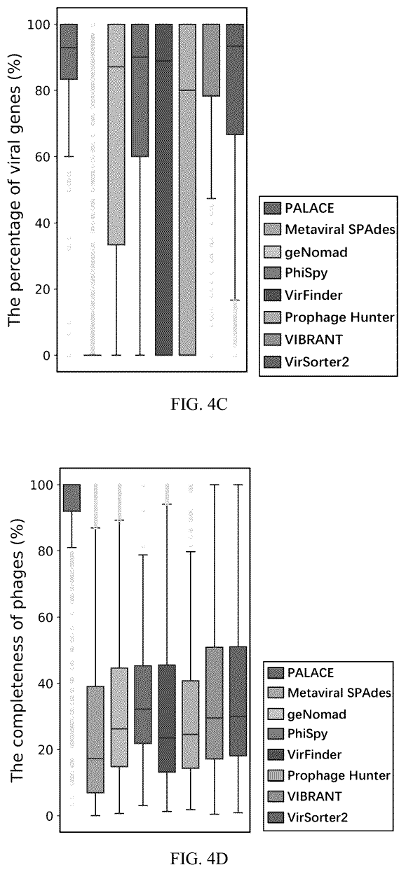

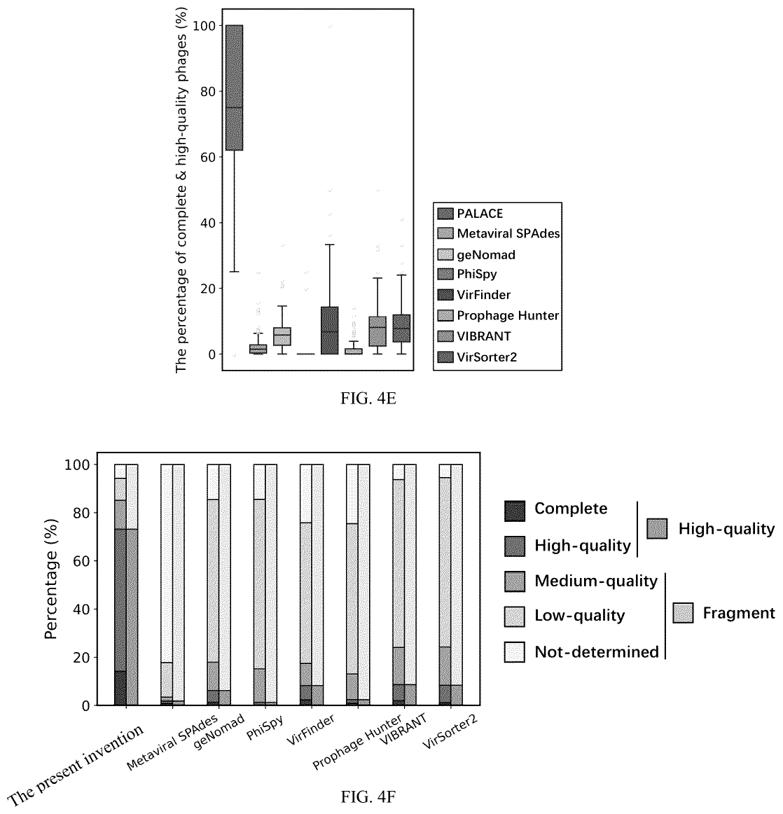

As shown in FIG. 4A, the length distributions of the phage genomes from all the methods are compared. The phages generated by PALACE exhibit a median length of 50.62 kb, which agrees with the median genome length of 52 kb observed in complete phages (Al-Shayeb et al. 2020). The benchmark tools produce phage genomes of median lengths ranging from 15.13 to 19.78 kb, which is noticeably shorter than the median genome length of complete phages.

Further, the quality of the generated phages is investigated using Check V (Nayfach, Camargo, et al. 2021). CheckV assesses the contamination and completeness of the genome by comparing queries with a comprehensive database of complete phage genomes. From the CheckV results, the present invention and PhiSpy generate the lowest percentage of prophages, i.e., phage sequences flanked by regions from bacterial genomes, with a median value of zero (FIG. 4B). Also, phages generated with the present method also exhibit the highest percentage of viral genes with a median value of 92.86% (FIG. 4C). The results demonstrate the successful exclusion of contigs of bacterial origin in the present method pipeline.

Additionally, careful examinations of the completeness of the phage genome with CheckV confirm the ability of the present method to assemble high-quality phages (FIGS. 4D-4F). The phage genomes assembled by the present method achieve the highest completeness, with a median value of 100% and a mean value of 88.84%, respectively, which significantly exceeds the second best, with a median value and a mean value of 32.15% and 38.09%, respectively. Among the 582 phages assembled with the present method, 73.02% genomes are classified as complete and high quality, demonstrating a significant improvement compared to 1.21-8.59% for the benchmark methods. These results indicate that existing methods for assembling or detecting phages from metagenomic data consistently generate phage genomes with a low level of completeness, whereas the present method assembles high-quality and confident phages effectively.

### Example 3. Assembling High-Quality Phage Genomes From Human Gut Microbiome

High-quality phage genome assembly enables a wide range of downstream phage studies. Inputting the human gut metagenomic data from three studies to the present method, the results show that there are 582 phages from ERP000108, 1,369 phages from ERP002061 (Nielsen et al. 2014, 225 human gut metagenome of stool samples of Danish and Spanish), and 2,799 phages from ERP003612 (Le Chatelier et al. 2013, 283 human gut metagenome of individuals from Denmark), with each phage assembly consisting of an average of eight contigs.

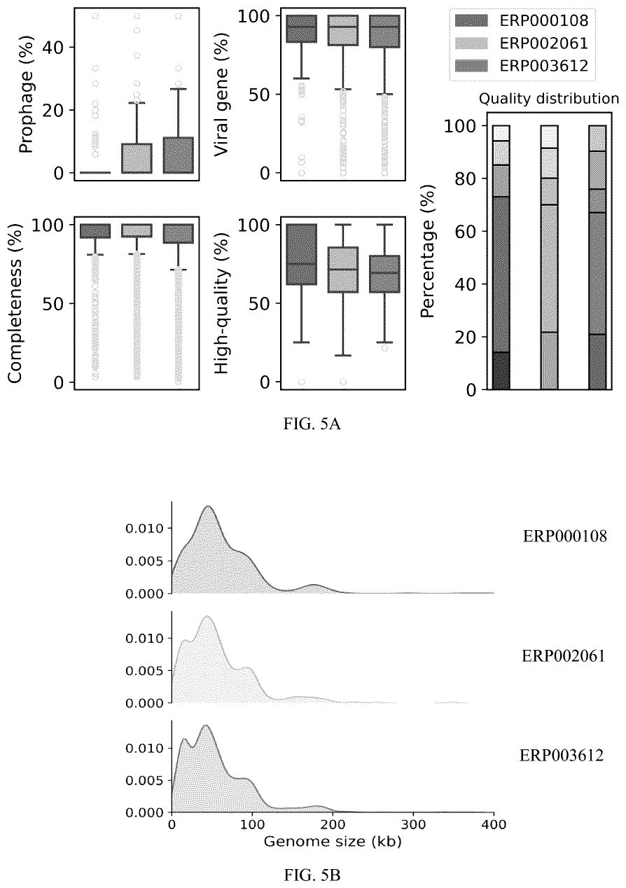

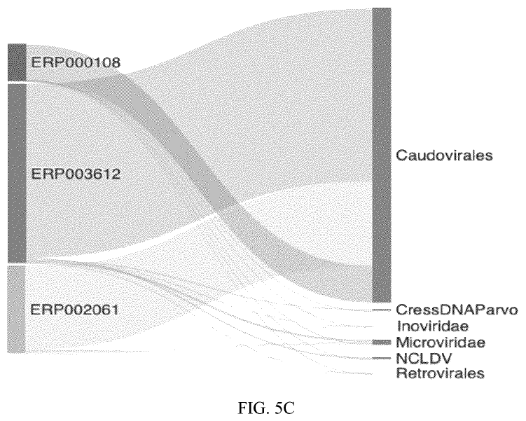

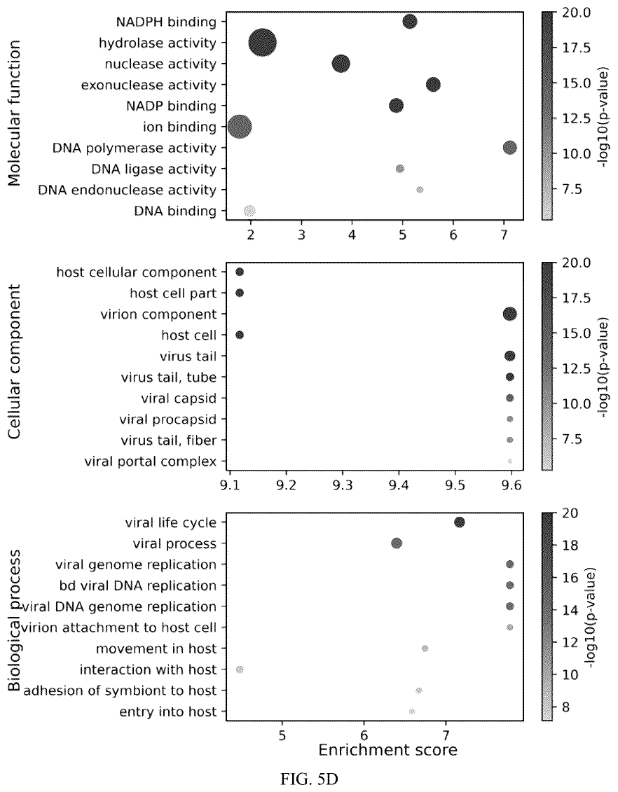

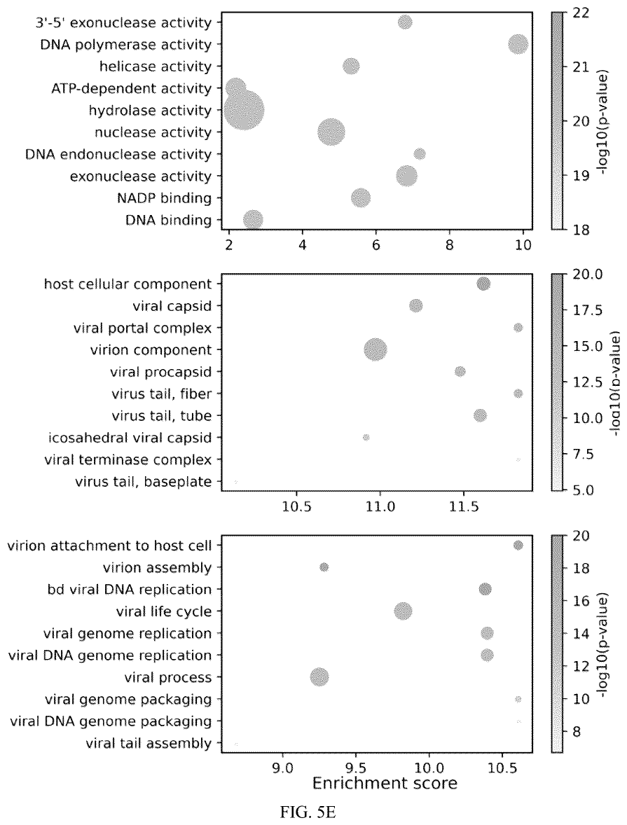

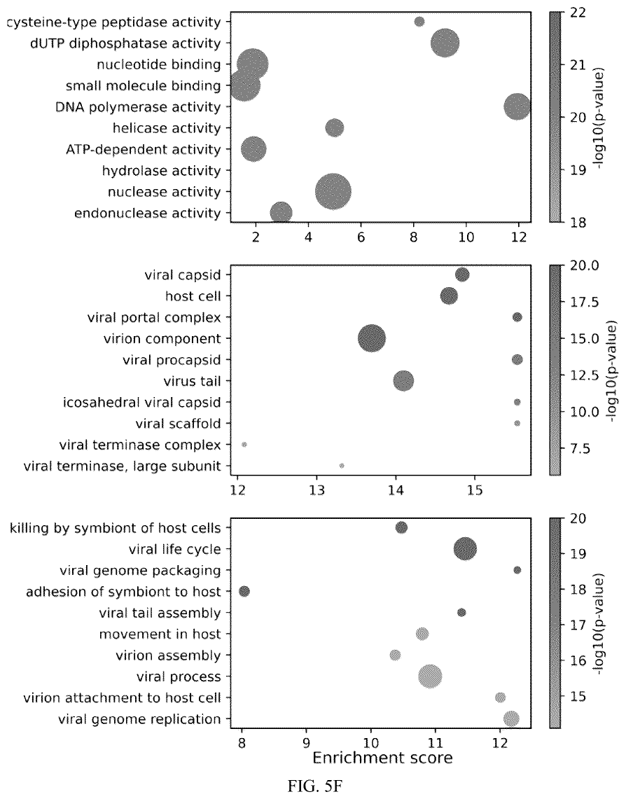

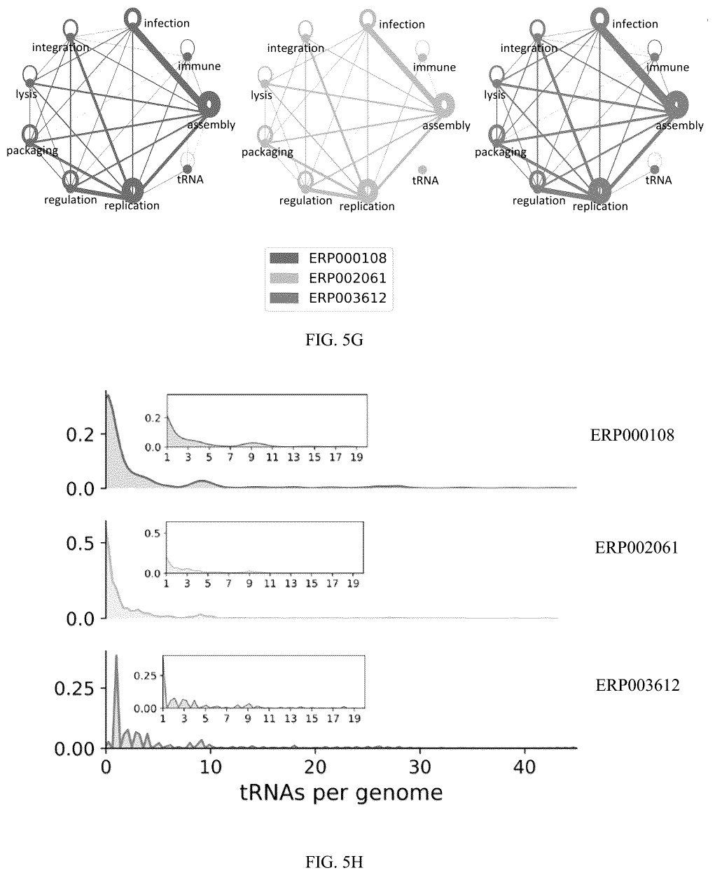

The quality of the assembled phage genomes is assessed with CheckV (FIG. 5A). First, <10% of the assembled phages are identified as prophages, and >90% of the genes are characterized as viral genes. These results demonstrate that the phage annotation process effectively excludes contamination of host genomes and collects the phage-origin contigs. Second, 67.06-73.02% of the assembled phages are identified as complete or high-quality, illustrating the successful assembly of high-quality phages from metagenomic data by the present invention.

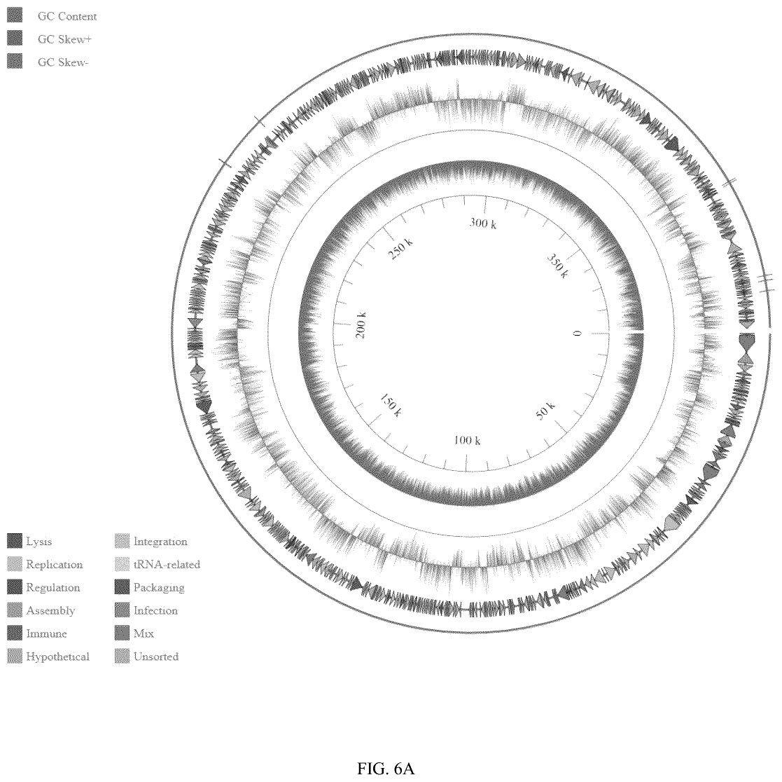

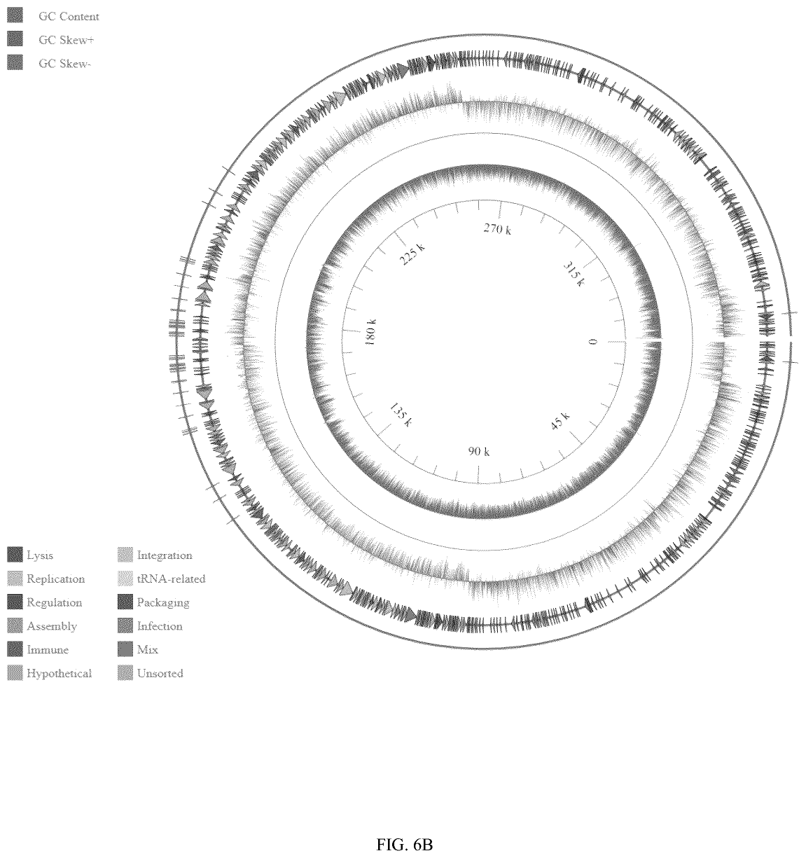

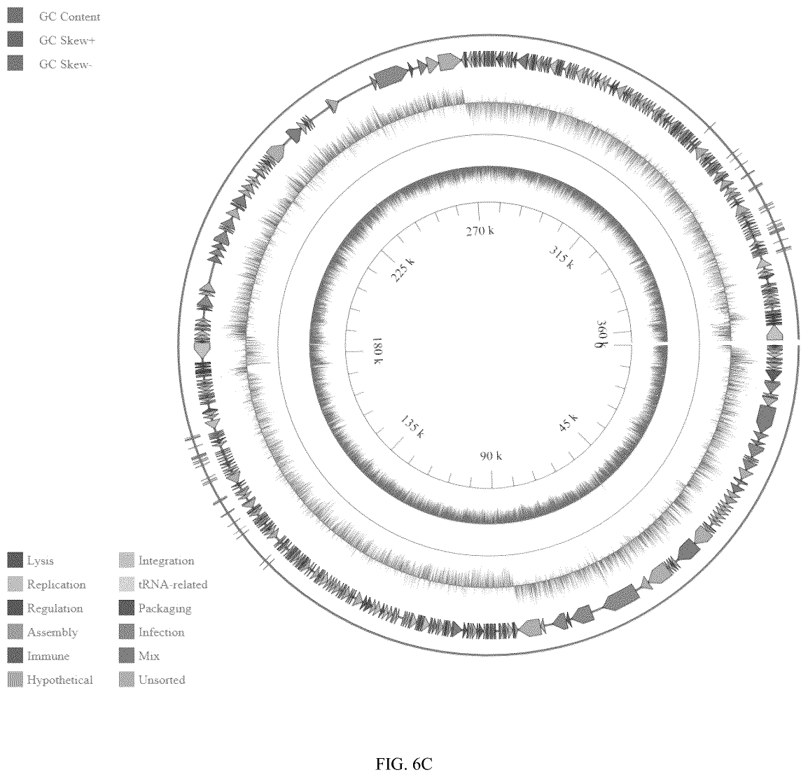

The assembled phages exhibit genome lengths ranging from 2.07 kb to 389.78 kb, with the majority falling within 10-100 kb, consistent with the genome length distribution of the sequenced phages (Russell and Hatfull 2017) (FIG. 5B). The present method assembles five, ten, and fifteen huge phage genomes (>200 kb) from ERP000108, ERP002061, and ERP003612, respectively. On average, each huge phage assembly comprises 33 metagenomic contigs. The genome maps for the largest phage genomes assembled from ERP000108 (FIG. 6A), ERP002061 (FIG. 6B), and ERP003612 (FIG. 6C) are provided.

Each assembled phage is assigned to a taxonomical group via homology search. Briefly, the viral taxonomic assignment is conducted with homology search. Leveraging a reference database that includes taxonomical annotations (Nayfach, Camargo, et al. 2021), the DIAMOND method (Buchfink, Xic, and Huson 2015) is employed to align the target phages to the reference database, facilitated through Check V with default parameter settings (Nayfach, Camargo, et al. 2021). Then, the viral taxonomies are assigned for the target phages based on the obtained homology results. The phages are annotated with six taxonomical groups, including Caudovirales, CRESS-DNA and Parvoviridae (CressDNAParvo), Inoviridae, Microviridae, nucleocytoplasmic large DNA virus (NCLDV), and Retrovirales (FIG. 5C). The majority of the assembled phages, with 538 from ERP000108, 1,244 from ERP002061, and 2,565 from ERP003612, are categorized into Caudovirales, which is known as the dominant phage taxonomy (Fokine and Rossmann 2014).

In addition, the functional proteins encoded by the assembled phages are explored. First, a Gene Ontology (GO) enrichment analysis is performed for the phage-encoded genes with the genes annotated from the metagenomes as the background (FIGS. 5D-5F). A collection of genes exhibits increased expression on phage genomes, demonstrating a significant enrichment of functional GO terms. Regarding molecular function, enriched GO terms include helicase activity (p-value<1×10⁻¹⁸), hydrolase activity (p-value<1×10⁻¹⁸), nuclease activity (p-value<1×10⁻¹⁸), etc., consistent with previous findings (Nayfach, Camargo, et al. 2021). Regarding the cellular component, the enriched GO terms specifically pertain to viral components, such as viral tail (p-value<1×10⁻¹⁸), viral capsid (p-value<1×10⁻¹⁸), and viral portal (p-value<1×10⁻¹⁸). Regarding the biological process, the enriched GO terms encompass various viral activities, such as viral life cycle (p-value<1×10⁻¹⁸), viral process (p-value<1×10⁻¹⁵), and viral DNA replication (p-value<1×10⁻¹⁵). These findings further verify the reliability of PALACE for assembling phages from metagenomic data.

Referring to FIG. 5G, the proteins are categorized into ten functional classifications and study their distributions on the phage genomes. Intriguingly, a consistent pattern is observed, wherein proteins from the same classification tended to be located adjacent to each other on the genomes (<5 kb). The relationship can also be established between proteins involved in infection and assembly, as well as regulation and replication. The results demonstrate that phage genomes exhibit a high degree of organization.

As shown in FIG. 5H, the tRNAs encoded by the assembled phages are examined. tRNAs are the predominant translation-associated genes observed in phages, attributed to their specific codon usage. In total, 10,636 tRNAs are identified from phages, with the highest number of tRNAs observed in a single phage being up to 46.

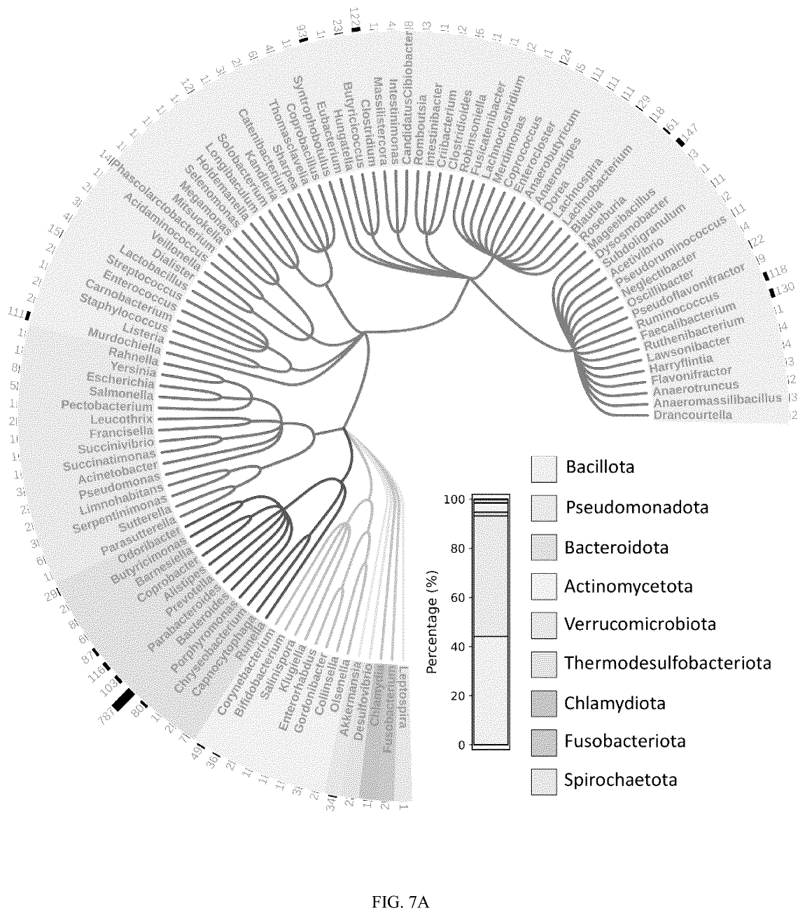

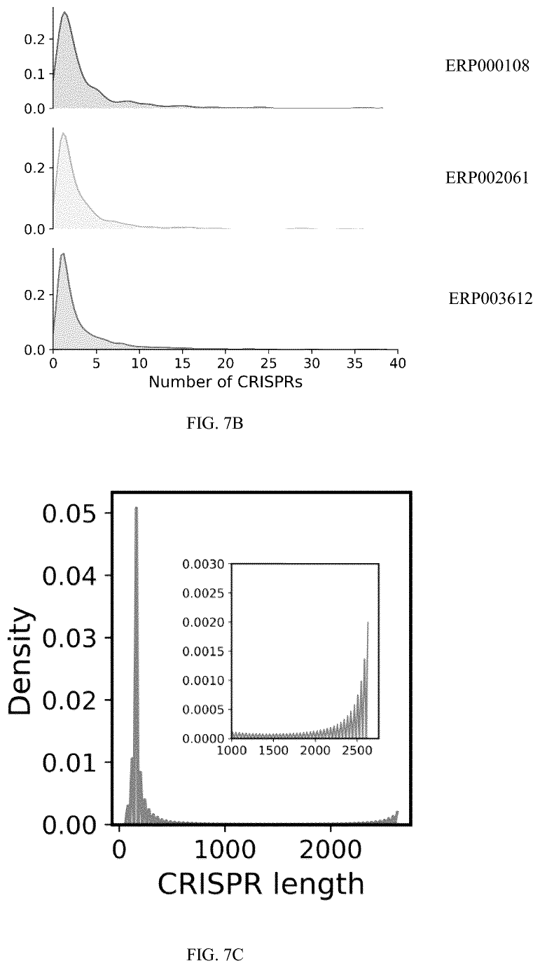

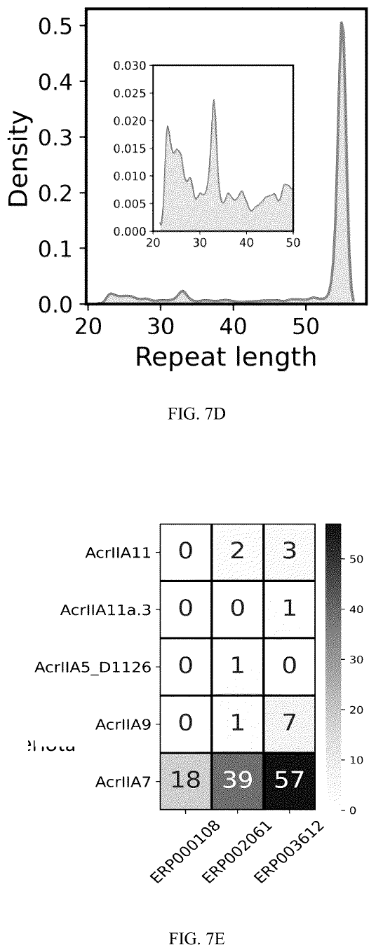

Furthermore, the phage-host interactions for the 4,750 phages assembled from metagenomic data are investigated. Identified with CRISPR-spacers, the hosts span across nine phylum taxonomies (FIG. 7A). Notably, the majority of phages exhibit a tendency to infect bacteria taxonomically classified under Bacillota and Pseudomonadota, whereas the bacterial genus Bacteroides, which serves as host for the highest number of phages, i.e., 787 phages, belongs to the phylum Bacteroidota. The CRISPR-Cas system and anti-CRISPR proteins play crucial roles in the interplay between phages and hosts. Bacteria deploy CRISPR-Cas systems to defend against phage infection, whereas phages have evolved countermeasures to evade the immune response. In addition, a phage has been reported to encode a CRISPR-Cas system to evade host innate immunity, adding complexity to the arms race between phages and bacteria (Seed et al. 2013). First, the CRISPR-Cas systems on the assembled phage genomes are identified. In total, 8,540 CRISPR arrays from the phages are detected (FIG. 7B), and the maximum number of CRISPRs observed in a single phage reaches up to 37. The distributions of the CRISPR length and repeat length for the identified CRISPR arrays are shown in FIG. 7C and FIG. 7D. Notably, there is no detection of Cas proteins on the phage genomes, suggesting a potential sharing of Cas proteins with their hosts. In addition, 129 anti-CRISPR proteins are identified from the phages, predominantly from the AcrIIA7 family, which is known to have a wide distribution across metagenomic data (Uribe et al. 2019) (FIG. 7E).

### Example 4. Exploring Gut Phage Community in CRC Patients

The microbiota in the human gut plays a crucial role in the genesis and progression of colorectal cancer (CRC). To explore the gut phage community in CRC patients, we applied the present invention to metagenomic data derived from healthy controls and CRC patients in two studies (Table 2). As a result, the present invention assembles 1,441 phages from FengQ_2015, including 557 from controls, 431 from adenoma patients, and 453 from CRC patients, along with 928 phages from YuJ_2015, including 362 from controls and 566 from CRC patients. On average, each phage is assembled from five contigs.

TABLE 2

| Study | # of samples | Description |
|---|---|---|
| FengQ_2015 | 56 controls, 46 CRCs, | Metagenomic shotgun- |
| | and 43 adenomas | sequencing on stool samples |
| YuJ_2015 | 52 controls and 72 CRCs | Metagenomic sequencing |
| | | on stool samples |

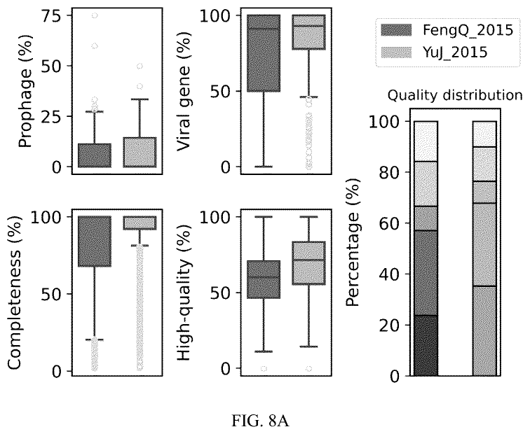

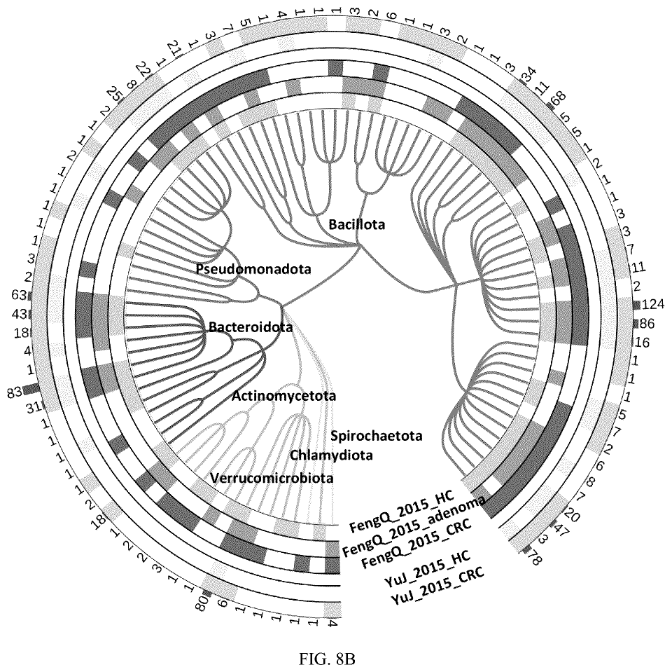

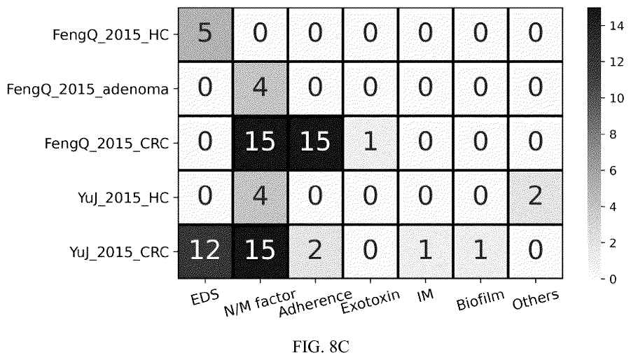

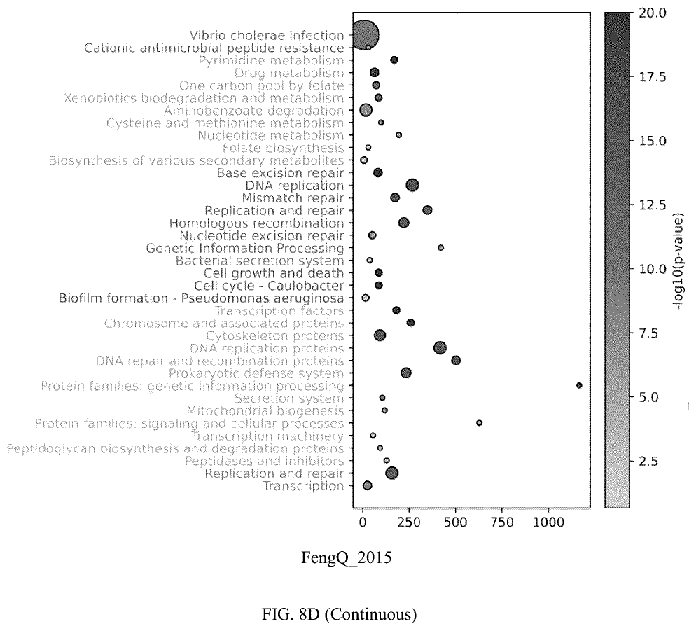

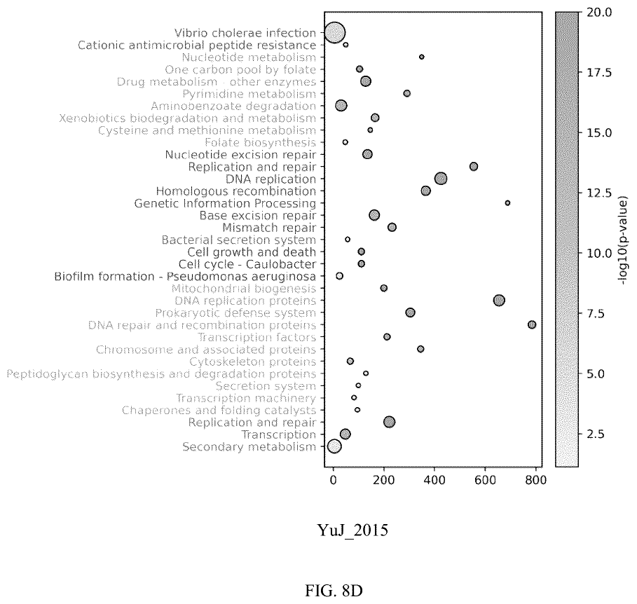

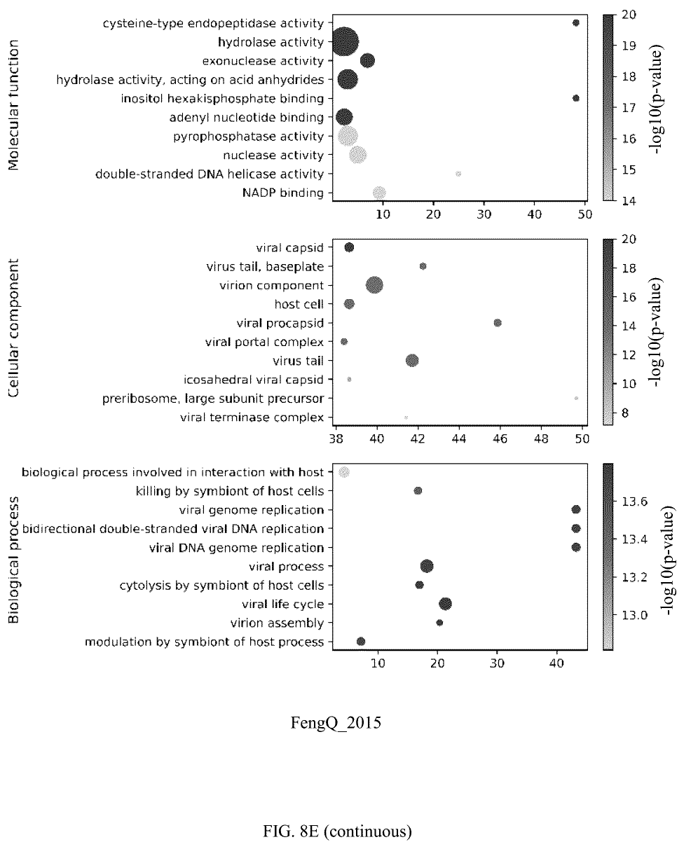

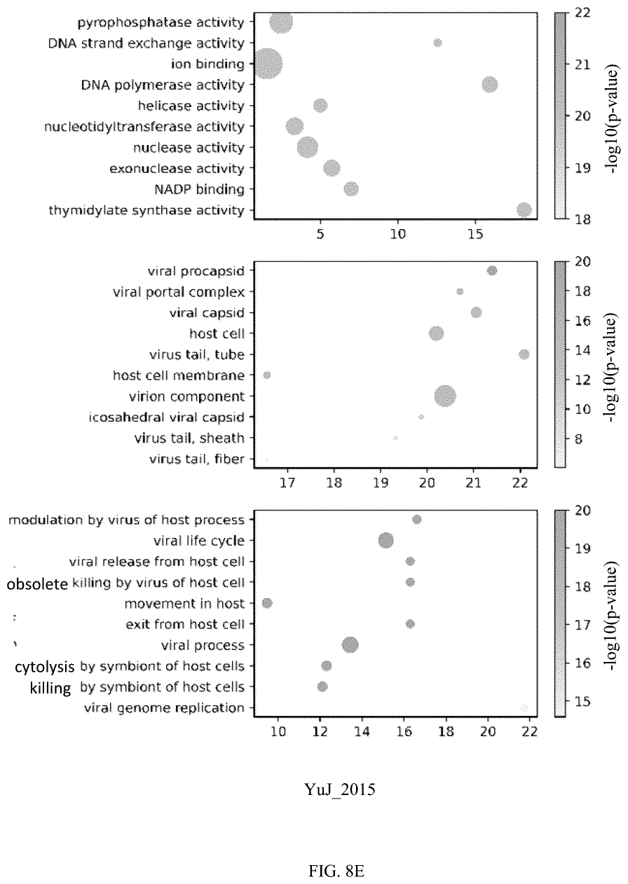

First, the quality of the phage genomes assembled by the present invention are evaluated (FIG. 8A). From CheckV results, the assembled sequences exhibit a high level of confidence as phage derived and a minimal presence of contamination from host sequences, with the median value of viral gene percentage as 91.98%. Also, the assembled phages achieve a median completeness value of 100%, with 57.04-67.78% identified as complete or high-quality phages.

Then, a GO enrichment analysis is conducted on the phage-encoded genes, using bacterial genes as the background. The results reveal an increased expression of GO terms associated with viral components and viral processes for the assembled phages, further verifying the reliability of the present method (FIG. 8E).

The hosts of the assembled phages encompass seven phylum taxonomies, with 49.7% originating from Bacillota and 35.05% from Bacteroidota (FIG. 8B). Specifically, 919 phages from healthy controls are identified to infect hosts from 57 bacterial genera, whereas the 1,019 phages from CRC patients exhibit a broader range of hosts, spanning 76 distinct bacterial genera. The observation agrees with the finding that the phage populations within the gut microbiomes of CRC patients are more diverse (Zuo, Michail, and Sun 2022).

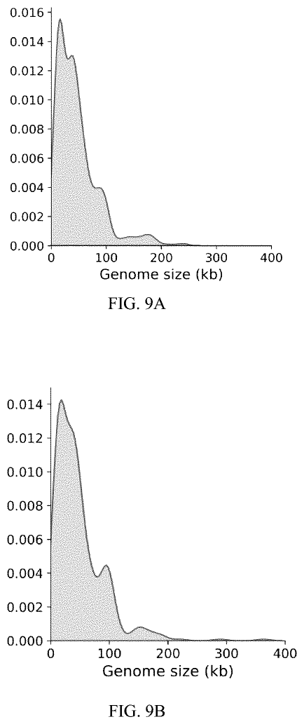

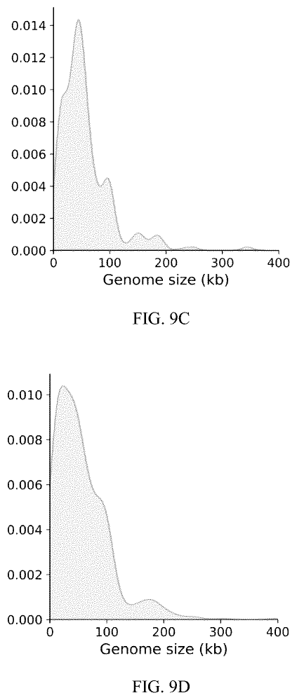

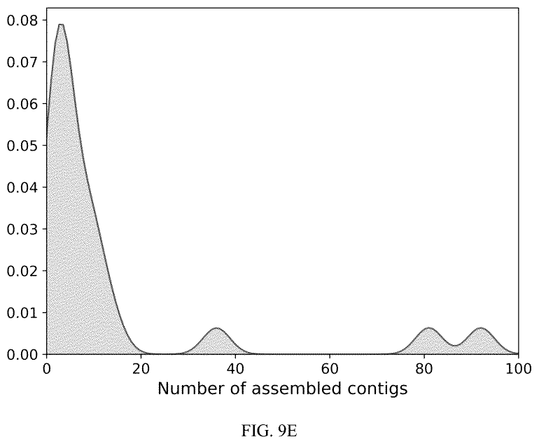

As shown in FIGs. 9A-9D, the length distributions of the assembled phage genomes from healthy controls and CRC patients are further examined. The genome lengths span a range of 2.68 kb to 397.24 kb, with a median length of 40.38 kb. Among these phages, there are 22 huge phage genomes (>200 kb), assembled with an average of fourteen contigs (FIG. 9E). There is no significant difference in the genome length distributions for phages assembled from controls and those from CRC patients, with a median length of 40.12 kb for phages from controls and 40.85 kb for phages from CRC patients. The distributions of tRNA and anti-CRISPR proteins do not exhibit noteworthy differences when comparing phages from healthy controls and those from CRC patients. Moreover, there is no significant differences in the distributions of tRNAs and anti-CRISPR proteins between the phages from healthy controls and CRC patients.

Next, it is investigated that the presence of the virulent factors on the phage genomes from healthy controls, adenoma patients, and CRC patients (FIG. 8C) and explore the discrepancies in their distributions. As a result, 11 virulent factors are detected among the 919 phages from controls and 62 virulent factors among the 1,019 phages from CRC patients. Specifically, a higher prevalence of nutritional and metabolic factors is observed in phages from CRC patients in both studies, which suggests that gut phages potentially contribute to shaping the dysbiotic state of the microbial ecosystem observed in CRC.

To further study the genetic features of phage genomes assembled from CRC patients, KEGG pathway enrichment analysis is performed for the functional proteins encoded by phages from CRC patients (FIG. 8D). Notably, the cationic antimicrobial peptide resistance pathway in the human diseases class is enriched in phages from CRC patients (p-value=2.29×10\hat{ }(−7)), indicating the potential involvement of phages in antimicrobial resistance mechanisms. Additionally, several significantly enriched pathways within the metabolism class are revealed, which aligns with the aforementioned observations. In genetic information processing and BRITE hierarchies classes, a notable enrichment of DNA replication and repair pathways is observed, suggesting a potential role of phages in responding to DNA damage in CRC patients. In the cellular processes class, the cell growth and death pathway demonstrate significant enrichment, indicating that phage may harbor genetic elements that interact with host cell signaling pathways involved in cell cycles.

In summary, the present invention provides a system and/or method for assembling high-quality phages from metagenomic data, which has been meticulously designed to address the four phage assembly challenges. First, through a combination of homology-based and deep learning-based methods, the present invention identifies contigs originating from both known and novel phage genomes, extending the assembly beyond the scope of existing references. The deep learning model, derived from the GP-GCN framework, accommodates the prevalent genomic variations present in phage genomes. Second, the present invention selectively includes the phage-derived contigs in the assembly process and implements rigorous filtering criteria to minimize bacterial contamination and ensure reliable phage assemblies. Third, the present invention aligns metagenomic reads to the phage-derived contigs and constructs a bipartite conjugate graph, where phage fragments are presented as vertices and aligned reads are presented as edges to concatenate the disjoint fragments. The present invention employs an iterative maximum matching algorithm to untangle the edges and form accurate and contiguous paths on the graph. Fourth, to overcome the varied coverage depths of phages and bacteria from metagenomic data, the present invention calculates the copy numbers of phage fragments and incorporates them into the assembly process, precisely preserving repeated sequences on the assemblies.

The assembly process of the present invention builds on conjugate graph theory, which offers a notable advantage in preserving the contiguity of phage genomes. The bipartite conjugate graph not only facilitates the reconstruction of complex phage genomes but also preserves the double helix DNA structure of the prevalent double-stranded phages. Accordingly, the maximum matching algorithm formulated is specifically designed to ensure that the double helix structure of assemblies is retained while concatenating the fragments. Extensions of the whole framework to the assembly of other genomes and genetic elements, such as bacterial genomes, viral genomes, and plasmids, from metagenomic samples are possible. The key is to tailor the strategy to specific characteristics of the target genetic entities.

For instance, when dealing with bacterial genomes, the framework needs to be customized to accommodate their larger genome size, as well as account for potential genomic rearrangements and horizontal gene transfer events. Similarly, the assembly of plasmids requires considering their circular nature and the inclusion of plasmid-specific genes to enhance assembly precision.

Applying the present invention to metagenomic data generates high-quality phages, providing a path to delve into the genetic structure of phages. One interesting observation is that proteins from the same function category exhibit a tendency to be co-located within close proximity on phage genomes. This consistent pattern can greatly assist in the phage annotation endeavors by establishing functional associations among neighboring proteins. Also, the high degree of organization intrinsic to phage genomes facilitates a more comprehensive understanding of the biological properties, action mechanisms, and evolutionary traits of phages.

From the phages assembled from the gut metagenomic samples of healthy controls and CRC patients, a notable enrichment of the antimicrobial peptide resistance pathway is observed in phages from CRC patients, suggesting that phages from CRC patients may have acquired or accumulated antimicrobial resistance genes, possibly through horizontal gene transfer events within the gut environment. The presence of the pathway raises concerns about the potential transfer of resistance traits to pathogenic bacteria, which may compromise the effectiveness of phage therapy. Therefore, careful consideration and exclusion of phages with the enriched antimicrobial resistance pathway is crucial in selecting and designing phage-based therapeutic strategies.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A machine learning (ML) based system with neural networks for reducing bacterial sequence contamination in constructing a phage genome by next-generation sequencing (NGS) dataset, comprising:
a phage NGS dataset;
a NGS reads screener configured to screen the phage NGS dataset and filter out low-quality NGS reads from the NGS dataset;
a NGS integrator configured to pre-assembles the filtered reads into contigs;
a contig classifier comprising an autoencoder based on a gapped pattern graph convolutional network (GP-GCN) for identifying an origin phage genome of the contigs so as to minimize a bacterial sequence contamination in the NGS dataset;
a graph generator configured to generate a copy-number-aware bipartite conjugate graph from the contigs; and
a phage sequence assembler configured to analyze the copy-number-aware bipartite conjugate graph to assemble the contigs into a potential phage sequence.

2. The machine learning based system of claim 1, wherein the autoencoder is trained with a phage genome dataset, so that the autoencoder is capable of matching the contigs to its origin phage genome.

3. The machine learning based system of claim 1, wherein the potential phage sequence is a linear or circular genome.

4. The machine learning based system of claim 1, wherein the copy-number-aware bipartite conjugate graph comprises vertices and edges, wherein the vertices are endpoints of each of the contigs and the edges are overlapping reads between each of the contigs.

5. The machine learning based system of claim 1, wherein the contig classifier further utilizes sequence homology and motif analysis to enhance the accuracy of identifying the origin phage genome of the contigs.

6. The machine learning based system of claim 1, further comprising a database management system configured to store and retrieve the phage NGS dataset, the filtered reads, the contigs, and the assembled phage sequences for future analysis and comparison.

7. The machine learning based system of claim 1, wherein the NGS reads screener, the NGS integrator, the contig classifier, the graph generator, and the phage sequence assembler are integrated into a unified software platform with a user-friendly graphical interface.

8. The machine learning based system of claim 1, further comprising a visualization module that provides graphical representations of the copy-number-aware bipartite conjugate graph and the assembled phage sequences.

9. The machine learning based system of claim 1, wherein the low-quality NGS reads are characterized by having an average quality score lower than 20, with more than 40% of the bases have a quality score less than 15, with more than 5′N′ bases, or being shorter than 15 bases.

10. A method of reducing a bacterial sequence contamination in phage genome constructions using NGS data analyzed by a machine learning system, comprising:
inputting a phage NGS dataset and filtering out low-quality NGS reads from the phage NGS dataset;
assembling the filtered reads into contigs;
training a machine learning model with a phage genome dataset and a bacteria genome dataset such that the trained ML model is able to identify and classify an origin phage genome of the contigs and reducing a bacterial sequence contamination in the phage NGS dataset;
generating a copy-number-aware bipartite conjugate graph based on the classified contigs; and
analyzing the copy-number-aware bipartite conjugate graph so as to assemble the contigs into potential phage sequences.

11. The method of claim 10, wherein the ML model comprises an autoencoder based on a GP-GCN.

12. The method of claim 10, wherein the ML model is trained with sequence homology and motif analysis features to enhance the accuracy of identifying and classifying the origin phage genome of the contigs.

13. The method of claim 10, wherein the potential phage sequence is a linear or circular genome.

14. The method of claim 10, wherein the copy-number-aware bipartite conjugate graph comprises vertices and edges, wherein the vertices are endpoints of each of the contigs and the edges are overlapping reads between each of the contigs.

15. The method of claim 10, further comprising storing and retrieving the phage NGS dataset, the filtered reads, the contigs, and the assembled phage sequences in a database for future analysis and comparison.

16. The method of claim 10, further comprising visualizing the copy-number-aware bipartite conjugate graph, and the assembled phage sequences through a graphical interface.

17. The method of claim 10, further comprising a step of fine-tuning the assembled phage sequences by comparing them against known phage databases and making adjustments to improve sequence accuracy.

18. The method of claim 10, wherein the low-quality NGS reads are characterized by having an average quality score lower than 20, with more than 40% of the bases have a quality score less than 15, with more than 5′N′ bases, or being shorter than 15 bases.
